(19) United States
(12) Patent Application Publication
(10) Pub. No.: US 2024/0058173 A1
(43) Pub. Date: Feb. 22, 2024

(54) MOLDABLE DECORATIVE SPLINT OR CAST MATERIAL AND METHODS OF MANUFACTURING

(71) Applicant: Bella Bling, LLC, Chelmsford, MA (US)

(72) Inventors: Cristina Watson, Chelmsford, MA (US); John Richard Bussiere, Littleton, MA (US)

(73) Assignee: Bella Bling, LLC, Chelmsford, MA (US)

(21) Appl. No.: 18/267,419

(22) PCT Filed: Dec. 13, 2021
(86) PCT No.: PCT/US21/63069

§ 371 Date: Jun. 14, 2023

Related U.S. Application Data
(60) Provisional application No. 63/125,348, filed on Dec. 14, 2020.

(51) Int. Cl. A61F 13/00; A61F 5/058; A61F 13/04

(52) U.S. Cl. CPC A61F 13/00059; A61F 5/058; A61F 13/041; A61F 13/00995

(57) ABSTRACT

Embodiments disclosed herein relate to moldable decorative splint materials and associated methods of manufacture. The splint material may include a plurality of decorative elements integrated with the splint material. The decorative elements may be interconnected within a network of flexible connectors via a plurality of supports. Each support of the plurality of supports may include a retaining edge that retains the decorative element within the support.

### RELATED APPLICATIONS

This application claims the benefit under 35 U.S.C. § 119(e) to U.S. Provisional Patent Application No. 63/125,348 filed Dec. 14, 2020, which is herein incorporated by reference in its entirety.

### FIELD

Disclosed embodiments relate to moldable decorative materials, for example for moldable splint or cast materials, and methods of manufacturing the same.

### DISCUSSION OF THE RELATED ART

Casts and splints are applied to patients to stabilize, immobilize and/or provide support to injured body parts. A heat-moldable sheet made of a low temperature thermoplastic may be used to form a splint or cast. Typically, the sheet is placed in heated water to heat the material until the thermoplastic becomes sufficiently pliable to be molded into a splint shape. A medical professional then molds the sheet to the portion of the patient being treated to form the splint or cast. As the material cools, it hardens to form the splint or cast.

### SUMMARY

According to one aspect, a method of manufacturing a decorative splint material is disclosed. The decorative splint material is adapted to become sufficiently moldable when heated such that the decorative splint material may be molded into a splint configuration that conforms to an anatomical body part and to harden when cooled such that the decorative splint material retains the splint configuration. The method includes heating a sheet formed from a heat-moldable base material to a first temperature and placing a network of flexible connectors onto the sheet. A plurality of discrete three-dimensional decorative elements are attached to the network of flexible connectors. The method further includes applying a force to the network of flexible connectors in a direction toward the sheet to at least partially embed the plurality of three-dimensional decorative elements in the sheet and cooling the sheet to a second temperature such that the network of flexible connectors is incorporated within the sheet with portions of the three-dimensional decorative elements being visible.

According to another aspect, a method of manufacturing a decorative splint material is disclosed. The decorative splint material is adapted to become sufficiently moldable when heated such that the decorative splint material may be molded into a splint configuration that conforms to an anatomical body part and to harden when cooled such that the decorative splint material retains the splint configuration. The method includes, heating a plurality of plastic pellets to a first temperature such that the pellets soften and form a softened sheet and placing a network of flexible connectors onto the softened mass. A plurality of discrete three-dimensional decorative elements are attached to the network of flexible connectors. The method further includes applying a force to the network of flexible connectors in a direction of the softened mass to at least partially embed the plurality of three-dimensional decorative elements in the mass such that at least a portion of the mass seeps through one or more openings within the network of flexible connectors and cooling the mass and the network of flexible connectors to a second temperature such that the network of flexible connectors is incorporated within the mass with portions of the three-dimensional decorative elements being visible.

According to yet another aspect, a moldable decorative splint material is adapted to become sufficiently moldable such that the decorative splint material may be molded into a splint configuration. The splint configuration may conform to an anatomical body part and harden such that the decorative splint material retains the splint configuration. The moldable decorative splint material may include a base material, a network of flexible connectors, and a plurality of supports. The plurality of supports may be attached to the network of flexible connectors, and a plurality of discrete three-dimensional decorative elements, each three-dimensional decorative element held within a support of the plurality of supports. At least a portion of each of the plurality of discrete three-dimensional decorative elements is visible.

According to a further aspect, a method is disclosed. The method includes attaching a plurality of supports to a network of flexible connectors and securing a plurality of discrete three-dimensional decorative elements within the supports via heat staking. Each of the plurality the supports covers a portion of a three-dimensional decorative element of the plurality of discrete three-dimensional decorative elements, and at least a portion of each of the plurality of the discrete three-dimensional decorative elements is not covered by the support to which the decorative element is secured.

According to an additional aspect, an apparatus includes a network of flexible connectors, a plurality of supports attached to the network of flexible connectors, and a plurality of discrete three-dimensional decorative elements. Each discrete three-dimensional decorative element of the plurality of discrete three-dimensional decorative elements is disposed within a respective support of the plurality of supports such that the respective support covers at least a portion of the three-dimensional decorative element and at least a portion of the three-dimensional decorative element is uncovered by its respective support.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a perspective view of a sheet of moldable material including decorative elements wrapped around a patient's wrist in a splint configuration according to one illustrative embodiment;

FIG. 2 is a perspective view of a network of flexible connectors holding decorative elements, the flexible connector and decorative elements being incorporated with a sheet of a base material according to one illustrative embodiment;

FIG. 3 is a top view of a network of flexible connectors holding decorative elements according to one illustrative embodiment;

FIG. 4 is a partial cross-sectional view of the network of flexible connectors and decorative elements taken along line 4-4 of FIG. 3;

FIG. 5 is a cross-sectional perspective view of a support and a three-dimensional decorative element according to one illustrative embodiment;

FIG. 6 is an exploded view of a manufacturing apparatus for making a sheet of moldable material including decorative elements according to one illustrative embodiment;

FIG. 7 is a top view of a sheet of moldable material including decorative elements according to one illustrative embodiment;

FIG. 8 is a flow chart of a method for manufacturing a sheet of moldable material including decorative elements according to one illustrative embodiment;

FIG. 9 is a flow chart of a method for manufacturing a sheet of moldable material including decorative elements according to another illustrative embodiment; and

FIG. 10 is a flow chart of a method for manufacturing a network of flexible connectors including decorative elements according to one illustrative embodiment;

FIG. 11 is a top view of a sheet of moldable material including decorative elements according to one illustrative embodiment.

### DETAILED DESCRIPTION

For ease of understanding, and without limiting the scope of the present disclosure, the devices to which this patent is addressed are disclosed below particularly in connection with splint materials and splints. For purposes herein, the term “splint material” means materials configured to be used in a splint, a cast, or other apparatus configured to support, stabilize, and/or immobilize an anatomical body part. Similarly, for purposes herein, the term “splint” means a splint, a cast, or other apparatus configured to support, stabilize, and/or immobilize an anatomical body part.

Splints are typically used when a person has been injured, has had surgery, or requires support, immobilization or stabilization for other medical reasons. Often, a patient may view the splint as unattractive and/or as a visual reminder of his or her injury. In some cases, these concerns may lead a patient to have a negative attitude regarding the splint, resist use of the splint, or perhaps request removal of the splint earlier than medically advisable.

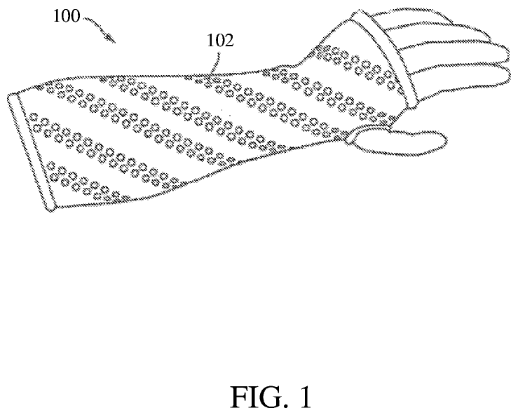

Embodiments disclosed herein provide a visually appealing splint material which may aid in persuading a patient to properly use the splint, and also may improve a patient's outlook regarding their medical condition. Additionally, by providing a variety of decorative splint material options, a patient may be provided with a choice as to the visual appearance of his or her splint, which may allow a patient to express their particular style or color preference, thereby giving their splint a personalized or customized look. For example, FIG. 1 shows an illustrative embodiment of a splint material formed around the arm of a patient adorned with decorative elements.

According to some embodiments, a heat-moldable splint material includes decorative elements, such as rhinestones, which are integrated with the splint or cast material prior to applying the splint to the patient. When the splint material is heated for shaping over a patient's body part, the decorative elements remain integrated with the material. Once the material cools and hardens, a visually attractive splint is formed.

In some embodiments, the decorative elements are embedded in a sheet of splint material. For example, rhinestones (or other decorative elements) may be embedded in the sheet of splint material during manufacture of the sheet. In other embodiments, a previously manufactured sheet of material may be heated to a softened state, and decorative elements may be embedded in the sheet by pressing the decorative elements into the sheet. The sheet may be cooled to a hardened state and stored and/or transported until needed for application to a patient. In still further embodiments, decorative elements may be attached to a sheet of splint material with an attachment element, such as a monofilament line.

By providing a sheet of splint material with previously integrated decorative elements, a patient may have a decorated splint applied without requiring a subsequent step of adding decorative elements to the splint.

In some embodiments, a network of flexible connectors may be incorporated within the splint material. For example, the network of flexible connectors may be formed from a series of filaments (e.g., plastic filaments, threads, cords, strands, or any other suitable configuration). The filaments may serve to interconnect a series of supports. For example, the filaments may be connected in a loose grid with the supports located at one or more intersections of the filaments. In some embodiments, a support may be located at each intersection of the filaments. The decorative elements may be affixed to the plurality of supports that are connected via the network of flexible connectors. In turn, the network of flexible connectors, with the decorative elements affixed to the supports, may be incorporated within the splint material by softening or melting the splint material, then adding the network of flexible connectors to the softened or melted splint material (e.g., by pressing the network of flexible connectors into the splint material). In some embodiments, the softened or melted splint material may seep through one or more openings in the network of flexible connectors.

In some embodiments, the supports include features which retain the three-dimensional decorative elements. For example, the supports may be cup shaped and each of the cup shaped supports may include a retaining edge which overlaps an upper edge of a decorative element to retain the decorative element within the support. The support may be heat staked or ultrasonically formed to crimp the retaining edge over the upper edge of the decorative element.

The retaining edges of the supports may serve to robustly retaining the decorative elements by providing an edge to abut a portion of the decorative element. Moreover, the abutment of the retaining edge against the decorative element may serve to retain the decorative element within the support when the support is exposed to the heated splint material when the network of flexible connectors equipped with supports is incorporated into the splint material.

To form the decorative splint material, a sheet formed from a base material (e.g., a heat moldable material for a splint) may be heated to first temperature to soften or melt the sheet. A network of flexible connectors (e.g., with decorative elements affixed to the network of flexible connectors as described above) may then be placed into the heated sheet. The network of flexible connectors may then be pressed into the heated sheet before the sheet is cooled such that the sheet hardens around the network of flexible connectors. In some instances, once the sheet is hardened, at least a portion of one or more of the decorative elements is visible from at least one side of the sheet. In some instances, the sheet may be formed by melting a series of pellets. The pellets may be formed from plastic or another suitable base material.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

FIG. 1 shows a decorated splint 100 wrapped around the wrist of a patient with integrated decorative elements 102 in a diagonal pattern. While embodiments disclosed herein may be used to integrate decorative elements 102 with the sheet of splint material before splint 100 is molded on the patient, additional decorative elements 102 may be added to splint 100 after splint 100 has been applied to the patient for customization of the design. For example, after splint 100 hardens, additional decorative elements 102 may be adhered to splint 100. In other embodiments, decorative elements 102 may be pressed into the splint material after the splint material has been molded over the patient, but before the material completely hardens.

A low temperature thermoplastic may be used to form a sheet of the splint material. Low temperature thermoplastics can be heated to a temperature at which the thermoplastic softens and becomes sufficiently pliable for molding the material into a desired shape. After cooling for a sufficient amount of time, the material hardens and maintains the desired shape. To soften low temperature thermoplastics, the material is typically heated to at least its melting point temperature using either heated water or heated air.

One example of a low temperature thermoplastic which may be used as splint material is poly epsilon caprolactone, which has a melting point temperature of approximately 62 degrees Celsius. According to some embodiments of the present disclosure, a thermoplastic having a melting point temperature between 50 degrees Celsius and 100 degrees Celsius inclusive may be used as at least a portion of the material for a sheet of splint material. In some embodiments, a low temperature thermoplastic having a melting point temperature between 62 degrees and 75 degrees inclusive may be used. In some embodiments, a low temperature thermoplastic with a melting point temperature of approximately 62 degrees Celsius may be used. Alternatively or additionally, other suitable splint materials (e.g., PCL Capa 6500) may be used to form the sheet, depending on the application.

Decorated splint material 100 may include any suitable type of decorative elements, including beads, crystals, gemstones, jewels, or rhinestones. Rhinestones may include artificial rhinestones such as glass rhinestones or plastic rhinestones, or any other suitable materials. Plastic rhinestones may be made of resin or acrylic plastic, or any other suitable materials. Other types of decorate elements may be used, depending on the application.

In some embodiments, the decorative elements may be three-dimensional. For example, in some instances, a three-dimensional decorative element embedded within decorated splint material 100 may have a height greater than the thickness of the base splint material. In some embodiments, the three-dimensional decorative elements may have a height of greater than 0.5 mm, a height greater than 1 mm, a height greater than 1.5 mm, or any other suitable height. In some embodiments, the three-dimensional decorative elements may have a height that is at least one-tenth of the largest lateral dimension of the decorative element.

In some embodiments, the decorative elements may be discrete from one another. Discrete decorative elements may be individually attached to a substrate using a suitable adhesive to create a desired pattern and/or design. The discrete decorative elements may be pre-attached to a substrate, such as a network of flexible connectors via a plurality of supports, as described in greater detail below.

The sheet of splint material may be formed in any suitable manner. For example, in some embodiments, the sheet of splint material is formed by melting or otherwise softening a plurality of pellets, such that the pellets may be formed into the sheet.

The thermoplastic sheets used for forming the splints disclosed herein may be provided in various colors, for example by including dyes within the heat-moldable material. In some embodiments, a sheet of splint material may have color added after the sheet of material has been manufactured, for example by painting the sheet.

The splint materials to which embodiments disclosed herein apply may be applied to a patient's arm, wrist, ankle, leg, nose or any other body part in need of support, stabilization and/or immobilization. In some embodiments, a sheet of splint material may be trimmed by a medical professional to a suitable size and shape according to the size and shape of the anatomical body part to be treated. In other embodiments, a sheet of splint material may be pre-cut or otherwise pre-shaped for a particular body part before being provided to the medical professional. In still further embodiments, a pre-cut or otherwise pre-shaped sheet may be provided, and the medical professional may perform additionally trimming to customize the sheet before heating.

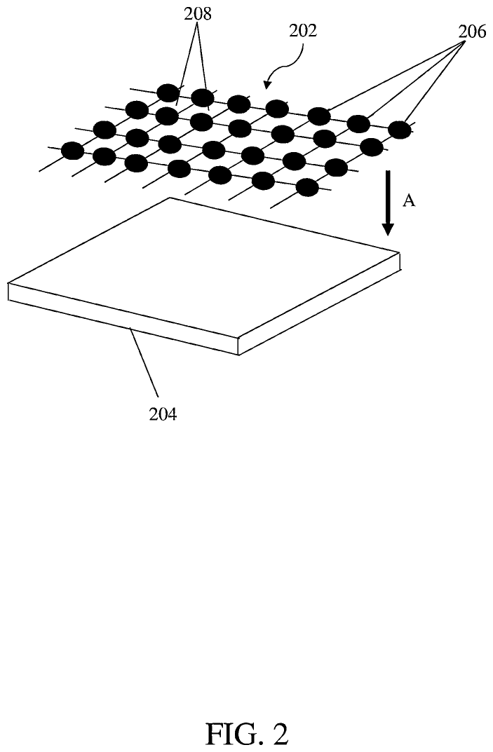

As shown in FIG. 2, the decorative elements 206 may be incorporated into a sheet of base material 204 (e.g., a splint material). For example, decorative elements 206 may be attached to a network of flexible connectors 202. In turn, sheet 204 may be heated (e.g., to a first temperature) such that network of flexible connectors 202 may be incorporated into sheet 204. Particularly, sheet 204 may be heated to a temperature beyond the melting point of the base material such that sheet 204 softens and/or liquifies. Network of flexible connectors 202 may then be placed into softened or liquified sheet 204. Specifically, a force may be applied to network of flexible connectors 202 in direction A (e.g., toward sheet 204). Network of flexible connectors 202 may include a plurality of openings 208. Portions of softened or liquified sheet 204 may seep through opening 208 in response to the force applied on the network of flexible connectors 202. Sheet 204 may then be cooled such that sheet 204 hardens around portions of network of flexible connectors 202, retaining network of flexible connectors 202 within sheet 204, forming a decorative splint material.

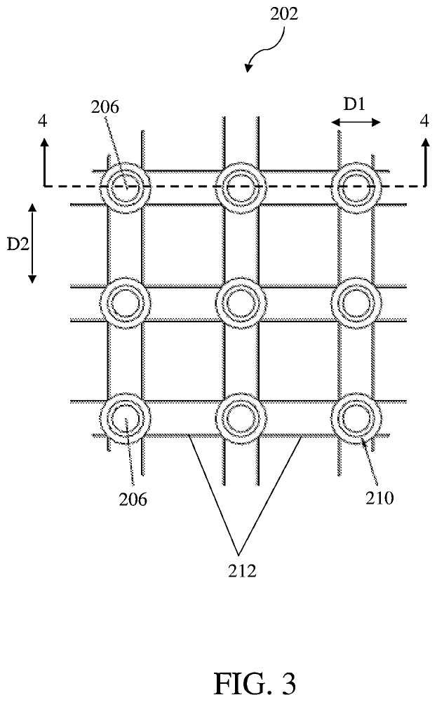

FIG. 3 shows a top view of network 202 of flexible connectors according to one illustrative embodiment. Within network 202 of flexible connectors, decorative elements 206 may be held within a series of supports 210. In turn, supports 210 may be attached to the network 202 of flexible connectors via one or more individual flexible connectors 212. In some embodiments (such as shown in FIG. 3), supports 210 are attached to the network of flexible connectors by a first pair of flexible connectors traveling in a first direction and a second pair of flexible connectors traveling in a second direction. The first and second directions may be perpendicular to each other. In some embodiments, supports 210 may be attached to the network of flexible connectors by a single flexible connector 212 or three or more flexible connectors 212. Flexible connectors 212 need not be attached to supports 210 in the cardinal direction as other orientations are also contemplated.

Alternatively or additionally, supports 210 may be attached to network of flexible connectors 202 via flexible connectors 212 in any suitable manner or configuration, depending on the application. In some embodiments, the network of flexible connectors 202 forms a mesh. Decorative elements 206 may be held to network of flexible connectors 202 that form a grid.

Supports 210 within network of flexible connectors 202 may have a diameter D1 of any suitable value. For example, in various embodiments, diameter D1 may be 1.5 mm or at least 1.5 mm, 2 mm or at least 2 mm, 2.5 mm or at least 2.5 mm, 3 mm or at least 3 mm, or any other suitable value. Further, in various embodiments, diameter D1 may be less than 4.5 mm, less than 4 mm, less than 3.5 mm, or any other suitable value. Combinations of the above referenced ranges are also contemplated. For example, distance D1 may be between 1.5 mm and 4.5 mm inclusive, between 2 mm and 4 mm, or 2.5 mm and 3.2 mm inclusive. Diameter D1 may be other suitable values or ranges, depending on the application.

Supports 210 may be spaced on network of flexible connectors 202 in a grid formation. Accordingly, supports 210 may be spaced a distance D2 apart from one another. Distance D2 may be any suitable value. For example, in various embodiments, distance D2 may be 7 mm or at least 7 mm, 7.5 mm or at least 7.5 mm, 8 mm or at least 8 mm, 8.5 mm or at least 8.5 mm, or any other suitable value. Further, in various embodiments, distance D2 may be less than 10 mm, less than 9.5 mm, less than 9 mm, or any other suitable value. Combinations of the above referenced ranges are also contemplated. For example, distance D2 may be between 7 mm and 10 mm inclusive, between 7.5 mm and 9.5 mm, or 8 mm and 9 mm inclusive. Distance D2 may be other suitable values or ranges, depending on the application.

Flexible connectors 212 may be flexible connectors of any suitable type. For example, flexible connectors 212 may be cords, stands, or any other suitable type of flexible connectors. Flexible connectors 212 may be made of any suitable material including nylon, flexible plastic, or any other suitable material.

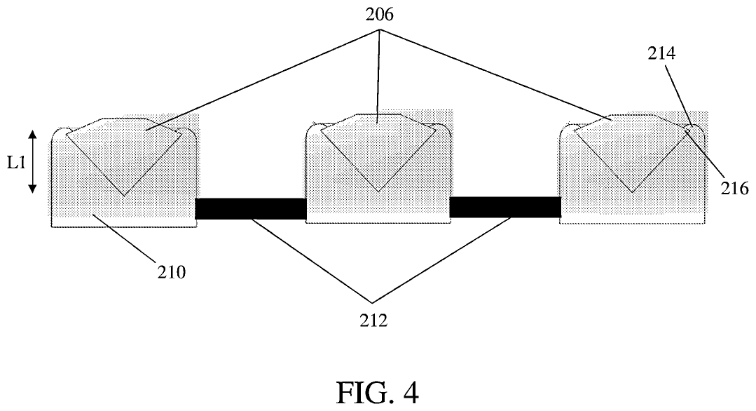

In some embodiments, flexible connectors 212 are affixed to supports 210 at a location below a depth of decorative elements 206. Specifically, in some embodiments, as shown by way of example in FIG. 4, decorative elements 206 may be embedded within supports 120 at a depth L1. In turn, flexible connectors 212 may be attached to supports 120 at a position vertically below depth L1. Alternatively, in some instances, each decorative element may be embedded within each support at a different distance or any combination of suitable distances, depending on the application.

Flexible connectors 212 may be so attached using any suitable manner of attachment. For example, in some embodiments, supports 210 are formed from a thermoplastic material. In such instances, flexible connectors 212 may be attached to supports 210 by first heating supports 210 such that supports 210 soften. Flexible connectors 212 may then be embedded into supports 210. Then, supports 210 may be cooled such that supports 210 reharden around flexible connectors 212, thus allowing supports 210 to be fixed to flexible connectors 212. In some embodiments, supports 210 include holes such that flexible connections 212 may be strung through the holes, thus connecting supports 210. Alternatively or additionally, flexible connectors 212 may be attached to supports 210 using other manners of fixing, including fasteners, adhesives, or any other suitable fixing means.

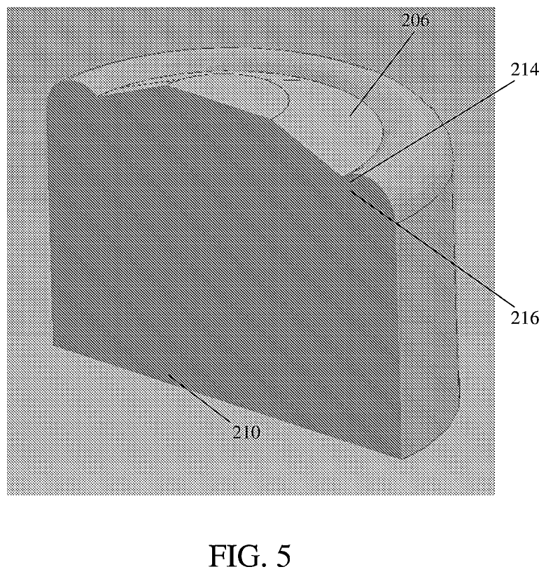

In some embodiments, supports 210 may include features that allow a support 210 to retain a decorative element 206 within support 210. For example, in some embodiments (e.g., the embodiments of FIGS. 4-5), support 210 includes a retaining edge 214 to secure decorative element 206 within support 210. Retaining edge 214 may cover a portion 216 of decorative element 206 to hold decorative element 206 within support 210. To fix decorative element 206 within support 210 via retaining edge 214, a portion of support 210 may be crimped over portion 216 of decorative element 206 to create retaining edge 214. Such crimping may be achieved by heat staking or ultrasonically forming retaining edge 214 over portion 216 of decorative element 206. Though any suitable manufacturing method may be employed to form retaining edge 214, depending on the application. In some embodiments, the support may be positioned over an edge of the decorative around an entire circumference of the decorative element.

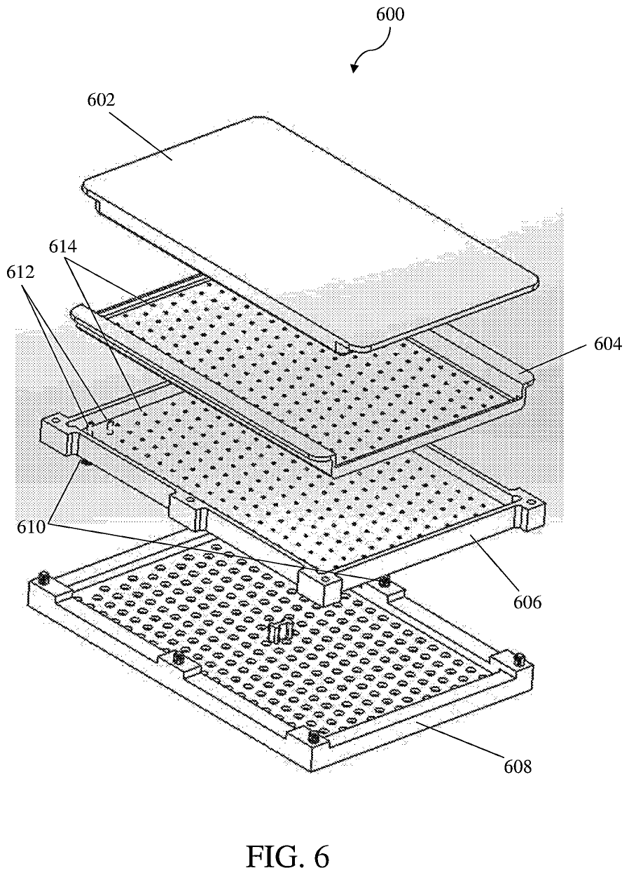

Decorated splint material 100 may be formed using any suitable apparatus. For example, FIG. 6 shows one embodiment of an apparatus 600 for manufacturing decorated splint material 100. Apparatus 600 may include a pressure plate 602, a release plate 604, a top base plate 606, and a bottom base plate 608. Top base plate 606 may nest within bottom base plate 608. In some embodiments, top base plate 606 includes a plurality of springs 610 located on the underside of bottom base plate 606. Top base plate 606 may be free to move relative to bottom base plate 608 when pressed toward bottom base plate 608. Springs 610 may be held in place by a corresponding plurality of pins 612 adapted to fit though openings 614 in top base plate 606. In turn, release plate 604 may be shaped to nest within top base plate 606. Moreover, release plate 604 may also include a plurality of openings 614 such that plurality of pins 612 may fit through opening 614 in release plate 604, thus holding release plate 604 in place relative to top base plate 606.

To manufacture decorated splint material 100, a base material, such as a sheet of suitable splint material or a large number of pellets may be placed within release plate 604. Release plate 604 may then be assembled with top base plate 606 and bottom base plate 608 as described above and then subsequently heated (e.g., in a convective oven). As the sheet and/or pellets of base material are heated, the base material softens and/or melts. Then, a network of flexible connectors, which may contain one or more decorative elements as described above, may be placed into release plate 604. In some instances, openings in the network of flexible connectors may correspond with pins 612, such that the network of flexible connectors adopts an orientation within release plate 604 corresponds with the orientation of pins 612. Subsequently, pressure plate 602 may be used to apply a force in the direction of release plate 604, which serves to incorporate the network of flexible connectors within the softened and/or melted base material. The apparatus is then cooled such that the base material hardens around the network of flexible connectors. Pressure plate 602 is then removed from apparatus 600 before release plate 604 is removed from top base plate 606.

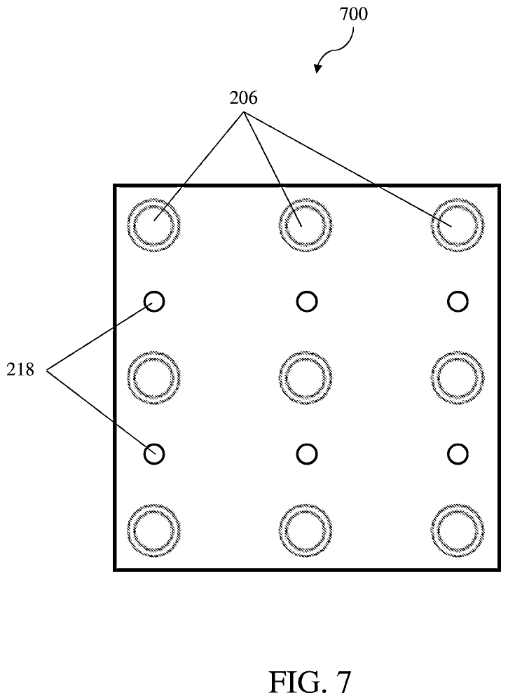

An example of such a finished product is shown in FIG. 7 as decorated splint material 700. As will be appreciated from FIG. 7, in some embodiments, one the network of flexible connectors (e.g., network of flexible connectors 202) is incorporated in the base material, at least a portion of decorative elements 206 is uncovered by the base material. Moreover, decorated splint material 700 may include one or more perforations 218. Perforations 218 may serve provide the splint material with breathability, for example, when the splint material is formed into a splint on a patient. Perforations 218 may be formed by pins 612, which may create perforations 218 when pressure plate 602 is placed over release plate 604. Alternatively or additionally, perforations 218 may be added after the sheet is manufactured, for example as described above, via drilling or any other suitable manner of making perforations. In some embodiments, such as the embodiment shown in FIG. 7, decorative elements 206 may be offset from perforations 218, though in other embodiments, perforations 218 may be positioned under decorative elements 206. In still other embodiments, a sheet of splint material may be provided without perforations, or the perforations may be provided only in certain sections of the sheet.

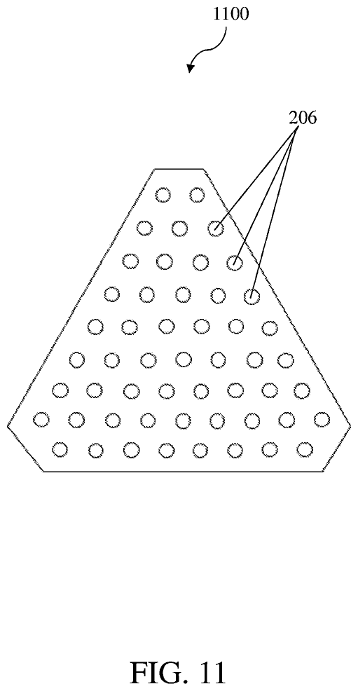

In some embodiments, such as the embodiment of FIG. 11, the decorative elements 206 may be positioned on the finished sheet 1100 such that the finished sheet 110 may be cut into any suitable shape, for example to fit on an appendage of a patient. Sheet 1100 may be arranged such that decorative elements 206 are similarly sized (e.g., circular with a diameter of 2 mm or 3 mm, or any other suitable diameter) and are equidistant from one another (e.g., 12 mm apart or any other suitable distance). Thus, in some embodiments, any three adjacent decorative elements 206 disposed on sheet 1100 may be in an approximately equilateral triangle configuration, allowing sheet 1100 to be cut into a triangular shape to fit over a nose of a patient. Other suitable configurations are also contemplated, depending on the application.

In some embodiments, pressure plate 602 is formed of carbon, nickel, a combination thereof or any other suitable material, depending on the application. In some embodiments, release plate 604, top base plate 606, and/or bottom base plate 608 may be formed of aluminum (e.g., 6061 Aluminum) or any other suitable material depending on the application.

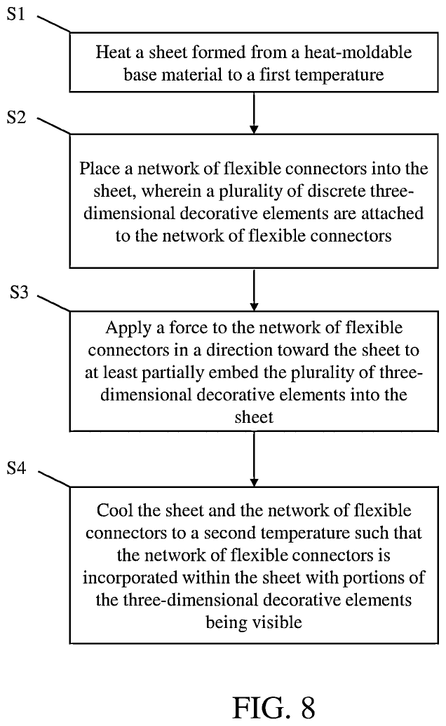

FIG. 8 is a flowchart showing an illustrative process for producing a decorated splint material (e.g., decorative splint material 700). At step S1, a sheet of base material (e.g., a heat moldable material) is heated to a first temperature, for example after being placed in release plate 604 as described above. The sheet may be heated to a sufficient temperature such that the sheet softens and/or melts. Then, at step S2, a network of flexible connectors containing a plurality of decorative elements (e.g., network of flexible connectors 202), may be placed onto the sheet, for example within release plate 604 as described above. At step S3, a force may be applied onto the network of flexible connectors in the direction of the sheet (e.g., direction A), for example via pressure plate 602, thus embedding the network of flexible connectors into the softened and/or melted sheet. At step S4, the combination of the network of flexible connectors and the sheet is left to cool, allowing the sheet to harden around the network of flexible connectors such that at least a portion of the decorative elements are visible through the sheet (e.g., in decorated splint material 700).

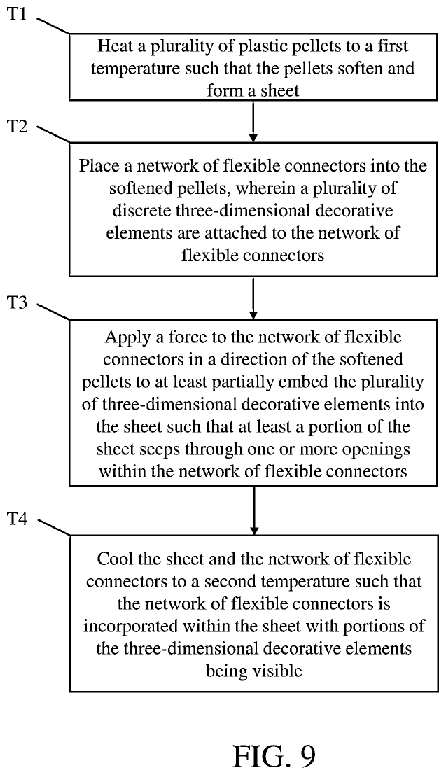

FIG. 9 is a flowchart showing yet another illustrative process for producing a decorated splint material (e.g., decorative splint material 700). At step T1, a plurality of plastic pellets of base material (e.g., a heat moldable material) are heated to a first temperature to form a softened mass. The softened mass may be in the form of a softened sheet of the base material, for example after being placed in release plate 604 as described above. The sheet may be heated to a sufficient temperature such that the sheet softens and/or melts. Then, at step T2, a network of flexible connectors containing a plurality of decorative elements (e.g., network of flexible connectors 202), may be placed onto the sheet, for example within release plate 604 as described above. At step T3, a force may be applied onto the network of flexible connectors in the direction of the sheet (e.g., direction A), for example via pressure plate 602, embedding the network of flexible connectors into the softened and/or melted sheet. At step T4, the combination of the network of flexible connectors and the sheet is left to cool, allowing the sheet to harden around the network of flexible connectors such that at least a portion of the decorative elements are visible through the sheet (e.g., in decorated splint material 700). In some embodiments, the first temperature is different from the temperature to which a clinician may heat the sheet to mold the sheet over an appendage of a patient.

When being heated to a sufficient temperature such that the sheet and/or pellets soften and/or melt, the sheet and/or pellets may be heated to a first temperature. The first temperature may be any suitable value. For example, in various embodiments, the first temperature may be 250 degrees Fahrenheit or at least 250 degrees Fahrenheit, 300 degrees Fahrenheit or at least 300 degrees Fahrenheit, 350 degrees Fahrenheit or at least 350 degrees Fahrenheit, 400 degrees Fahrenheit or at least 400 degrees Fahrenheit, or any other suitable value. Further, in various embodiments, the first temperature may be less than 550 degrees Fahrenheit, less than 500 degrees Fahrenheit, less than 450 degrees Fahrenheit, or any other suitable value. Combinations of the above referenced ranges are also contemplated. For example, first temperature may be between 250 degrees Fahrenheit and 550 degrees Fahrenheit inclusive, between 300 degrees Fahrenheit and 500 degrees Fahrenheit, or 350 degrees Fahrenheit and 450 degrees Fahrenheit inclusive. The first temperature may be other suitable values or ranges, depending on the application.

When being heated to a sufficient temperature such that the sheet and/or pellets softens and/or melts, the sheet and/or pellets may be heated for a first time period. The first time period can be any suitable value. For example, in various embodiments, the first time period may be 4 minutes or at least 4 minutes, 4.5 minutes or at least 4.5 minutes, 5 minutes or at least 5 minutes, 5.5 minutes or at least 5.5 minutes, or any other suitable value. Further, in various embodiments, the first time period may be less than 7 minutes, less than 6.5 minutes, less than 6 minutes, or any other suitable value. Combinations of the above referenced ranges are also contemplated. For example, first time period may be between 4 minutes and 7 minutes inclusive, between 4.5 minutes and 6.5 minutes, or between 5 minutes and 6 minutes inclusive. The first time period may be other suitable values or ranges, depending on the application.

When being cooled such that the sheet hardens, the sheet may be cooled for a second time period. The second time period may be any suitable value. For example, in various embodiments, the second time period may be 8.5 minutes or at least 8.5 minutes, 9 minutes or at least 9 minutes, 9.5 minutes or at least 9.5 minutes, 10 minutes or at least 10 minutes, or any other suitable value. Further, in various embodiments, the second time period may be less than 11.5 minutes, less than 11 minutes, less than 10.5 minutes, or any other suitable value. Combinations of the above referenced ranges are also contemplated. For example, first time period may be between 8.5 minutes and 11.5 minutes inclusive, between 9 minutes and 11 minutes, or between 9.5 minutes and 10.5 minutes inclusive. The second time period may be other suitable values or ranges, depending on the application.

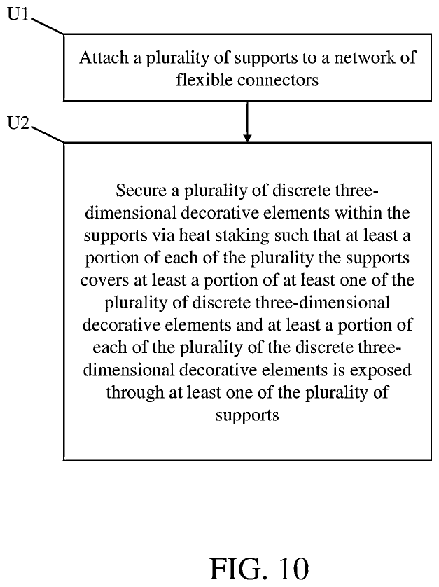

FIG. 10 is a flowchart showing an exemplary process for producing a network of flexible connectors with integrated decorative elements. At step U1, a plurality of supports (e.g., supports 210) may be attached to a network of flexible connectors (e.g., network of flexible connectors 202). As described above, the network of flexible connectors may be embedded within the plurality of supports by first heating the supports such that the supports soften and/or melt, incorporating the flexible connectors into the support, and then allowing the support to cool and reharden. Then, at step U2, the decorative elements are attached to the supports. The supports may be once again softened (e.g., via heating) and the decorative elements placed into the supports. The supports may then be allowed to cool and reharden to fix the decorative elements within the support. Subsequently, portions of the supports may be heat staked to crimp the portions of the supports over portions of the decorative elements such that the portions of the supports over portions of the decorative elements, retaining the decorative elements within the support. Particularly, heat staking may serve to limit or eliminate the use of adhesives to attach the decorative elements to their supports. Adhesives may lose their binding properties when supports 210 become heated during the embedding into the sheet of base material and/or during molding. Alternative methods of crimping may instead be employed including ultrasonic forming or any other suitable crimping process.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method of manufacturing a decorative splint material, wherein the decorative splint material is adapted to become sufficiently moldable when heated such that the decorative splint material may be molded into a splint configuration that conforms to an anatomical body part and to harden when cooled such that the decorative splint material retains the splint configuration, the method comprising:
heating a sheet formed from a heat-moldable base material to a first temperature;
placing a network of flexible connectors onto the sheet, wherein a plurality of discrete three-dimensional decorative elements are attached to the network of flexible connectors;
applying a force to the network of flexible connectors in a direction toward the sheet to at least partially embed the plurality of three-dimensional decorative elements in the sheet; and
cooling the sheet to a second temperature such that the network of flexible connectors is incorporated within the sheet with portions of the three-dimensional decorative elements being visible.

2. The method of claim 1, wherein each of the three-dimensional decorative elements is held by a respective support, and the supports are attached to the flexible connectors.

3. The method of claim 1, wherein each of the three-dimensional decorative elements is secured to its respective support via heat staking.

4. The method of claim 1, wherein applying the force to the network of flexible connectors in the direction of the sheet includes allowing at least a portion of the sheet to seep through one or more openings within the network of flexible connectors.

5. The method of claim 1, wherein at least partially embedding the network of flexible connectors into the sheet includes embedding the network of flexible connectors such that at least a portion of each of the plurality of discrete three-dimensional decorative elements is not covered by the sheet.

6. The method of claim 1, wherein applying the force to the network of flexible connectors in the direction of the sheet includes pressing on the network of flexible connectors with a pressure plate.

7. The method of claim 1, wherein prior to heating the sheet formed from the heat-moldable base material to a first temperature, the sheet is placed on a base plate.

8. The method of claim 1, wherein the discrete three-dimensional decorative elements comprise beads.

9. The method of claim 1, wherein the discrete three-dimensional decorative elements comprise rhinestones.

10. The method of claim 1, wherein the network of flexible connectors comprises a mesh.

11. The method of claim 1, wherein the flexible connectors comprise cords.

12.-23. (canceled)

24. A moldable decorative splint material adapted to become sufficiently moldable such that the decorative splint material may be molded into a splint configuration that conforms to an anatomical body part and to harden such that the decorative splint material retains the splint configuration, the moldable decorative splint material comprising:
a base material;
a network of flexible connectors;
a plurality of supports attached to the network of flexible connectors; and
a plurality of discrete three-dimensional decorative elements, each three-dimensional decorative element held within a support of the plurality of supports;
wherein at least a portion of each of the plurality of discrete three-dimensional decorative elements is visible.

25. The moldable decorative splint material of claim 24, wherein at least a portion of each of the plurality of discrete three-dimensional decorative elements extends out of the base material.

26. The moldable decorative splint material of claim 24, wherein the network of flexible connectors includes one or more openings configured to allow the base material to seep through the one or more openings.

27. The moldable decorative splint material of claim 24, wherein each support of the plurality of supports is cup shaped.

28. The moldable decorative splint material of claim 24, wherein the discrete three-dimensional decorative elements comprise beads.

29. The moldable decorative splint material of claim 24, wherein the discrete three-dimensional decorative elements comprise rhinestones.

30. The moldable decorative splint material of claim 24, wherein the network of flexible connectors comprises a mesh.

31. The moldable decorative splint material of claim 24, wherein the flexible connectors comprise cords.

32. The moldable decorative splint material of claim 24, wherein the flexible connectors comprise strands.

33. The moldable decorative splint material of claim 27, wherein each of the cup shaped supports includes a retaining edge which overlaps an upper edge of a decorative element to retain the decorative element within the support.

34. The moldable decorative splint material of claim 33, wherein the support is heat staked or ultrasonically formed to crimp the retaining edge over the upper edge of the decorative element.

35.-56. (canceled)
